(19) United States
(12) Patent Application Publication
(10) Pub. No.: US 2010/0304419 A1
(43) Pub. Date: Dec. 2, 2010

(54) METHOD FOR DETERMINING THE PROGNOSIS OF GASTRIC CANCER

(75) Inventors: Hsueh-Fen Juan, Taipei City (TW); Chiung-Nien Chen, Taipei City (TW); Chien-Wei Tseng, Taipei City (TW); King-Jen Chang, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/612,904

(22) Filed: Nov. 5, 2009

(30) Foreign Application Priority Data
May 27, 2009 (TW) 098117727

(51) Int. Cl. C12Q 1/02; G01N 33/574; G01N 33/53; G01N 33/68; C07K 16/00

(52) U.S. Cl. 435/29; 436/64; 436/501; 436/86; 530/389.1; 204/543

(57) ABSTRACT

The 14-3-3β protein is used herein as a tumor marker for prognosis in gastric cancer. The method comprises steps of providing a biological sample, qualifying the 14-3-3β protein level in the sample, and comparing the 14-3-3β protein level in the sample with a normal sample. Upon the 14-3-3β protein level in the biological sample is higher than in the normal sample, which represents the patient providing the sample has a poor prognosis. Having the higher sensitivity and specificity, 14-3-3β protein can be used as a tumor marker for prognosis of gastric cancer.

### FIELD OF THE INVENTION

The present invention relates to a method of prognosis, especially a method for determining the prognosis of gastric cancer.

### BACKGROUND OF THE INVENTION

Gastric cancer is one of the most important health threats today, which ranks as the 4th commonly diagnosed cancer with the second highest mortality rate according to WHO. It can be classified as early gastric cancer and advanced gastric cancer. This cancer is often asymptomatic or causes only nonspecific symptoms in its early stages. In the past, 80% of the patients were diagnosed as advanced tumors at the time of diagnosis, and recurring of cancer was common after surgery. According to previous reports, patients with stage I disease have a good prognosis, and those with stage IV disease show a very poor prognosis. The five-year survival rate is less than 35% in the late stages from previous reports. Therefore early diagnosis of gastric cancer to extend the survival rate is very important. Among them, serological tumor marker available for detection and prognostic evaluation of early and symptom-less gastric cancer is a simple, cost effective method.

Many tumor markers for gastric cancer have been found in recent years such as carcinoembryonic antigen (CEA), CA19-9 (carbohydrate antigen), CA72-4 (carbohydrate antigen 72-4). However, these markers have limited sensitivity and specificity, which cause mistakes and difficulties in diagnosis. Due to the current limitation to early diagnosis of gastric cancer, it is of great value to identify new potential biomarkers with high sensitivity and specificity.

The 14-3-3 proteins are a large family of approximately 25-30 kDa acidic proteins which exist primarily in the brain and neural system, also found in heart, liver, kidney, intestine, and testis. They can also be detected at the cell membrane and in intracellular organelles such as the nucleus, the Golgi apparatus, mitochondria, and chloroplast. There are at least seven isoforms, β, γ, ε, σ, δ, τ and η that have been identified in mammals. α and δ are the phosphoforms of β and γ respectively. The 14-3-3β protein is involved in many functions including the regulation of metabolism, cell-cycle control, signal transduction, apoptosis, protein trafficking, transcription, stress responses, and malignant transformation. It was known to bind with more than 200 receptors, covers with almost all the cellular reactions. Though 14-3-3β protein was related to several diseases such as Alzheimer's or Parkinson's disease, only 14-3-3σ was involved in development of epithelial cancers such as breast and gastric cancer.

### SUMMARY OF THE INVENTION

Due to the limitation of current cancer markers in gastric cancer, there is an urgent need to identify new potential biomarkers with high sensitivity and specificity.

A primary objective of the present invention is to provide a method for determining the prognosis of gastric cancer.

Another objective of the present invention is to provide a method of screening a compound for inhibiting the expression of 14-3-3β protein.

Yet another objective of the present invention is to provide a kit, which comprises an effective dose of an anti-14-3-3β protein antibody for detecting the risk for developing tumor invasion or distal metastasis of gastric cancer.

To overcome the problems of the known technology and fulfill the objective of the present invention, 14-3-3β protein was used as tumor marker for detecting gastric cancer, which comprises the steps of: providing a biological sample; determining an expression level of 14-3-3β protein in the biological sample; and comparing the expression level of 14-3-3β protein in the biological sample with a reference expression level of 14-3-3β protein in a normal sample; wherein a poor prognosis is indicated if the expression level of 14-3-3β protein in the biological sample is higher than the reference expression level of 14-3-3β protein of the normal sample.

The technology used in the present invention further comprises a method of screening a compound for inhibiting the expression of 14-3-3β protein in a biological sample, comprising: contacting a test compound with a biological sample expressing 14-3-3β protein; detecting the expression level of 14-3-3β protein in the biological sample; and selecting the test compound that inhibits the expression level of 14-3-3β protein in the biological sample as compared to the expression level of 14-3-3β protein detected in the absence of the test compound.

In addition, a kit can be developed based on the above-mentioned methods, which comprises an effective dose of an anti-14-3-3β protein antibody for detecting the risk for developing tumor invasion or distal metastasis of gastric cancer.

The 14-3-3β protein was proved to be an important cancer marker closely related to gastric cancer in the present invention. A sensitivity of 83% for 14-3-3β protein is higher than that for CEA (33%). And 14-3-3β protein is found to be a more reliable cancer marker than CEA in gastric cancer diagnosis since it showed a higher accuracy in prediction for survival rate than that of CEA. In addition, using 14-3-3β protein as a serologic cancer marker has the advantages of simple and cost saving, which can be applied as a diagnostic tool in detecting gastric cancer at an early stage and prognosis after surgery.

The compound of the present invention for inhibiting 14-3-3β protein expression can be obtained from many combinatorial chemical databases. These databases include peptides, peptoids, peptidomimetics, nucleic acids, small molecules or other drugs. Gastric cancer cell line can be used as gastric cancer if the method is performed ex vivo. Proper gastric cancer cell lines include AGS, KATOIII, TSGH, SC-M1 and N87. The expression level of 14-3-3 β protein can be measured in vivo or ex vivo. For example, the expression level of 14-3-3β protein can be determined through a biological sample such as a solid tumor tissue, excrement, blood or digestive fluid of each individual before or after the compound treatment. The method for determining the expression level of 14-3-3β protein was described below. The curing ability of the test compounds can be determined after measuring the effects of compounds on tumor size, growth or migration in a subject.

The present invention is further explained in the following embodiment illustration and examples.

### BRIEF DESCRIPTION OF THE DRAWINGS

FIGS. 1A to 1D show changes of 14-3-3β protein expression level in cell line SC-M1 after 5-FU (5-fluorouracil) treatment for 48 hours by using proteomic approaches and Western blot;

FIGS. 2A to 2E show in situ staining and Western blot analysis of 14-3-3β protein from normal tissue (N) and gastric cancer tissue (T) samples;

FIG. 3A shows the plasma 14-3-3β protein levels in patients with gastric cancer (Stage I to Stage IV) and normal individuals by using ELISA;

FIG. 3B shows the correlation between 14-3-3β protein values and survival rates in stage I gastric cancer patients with high 14-3-3β protein levels and with low 14-3-3β protein levels;

FIG. 3C shows the plasma CEA levels in patients with gastric cancer (Stage I to Stage IV) and normal individuals;

FIGS. 4A to 4E show the ROC curves of 14-3-3β protein and CEA in patients with stage I˜IV gastric cancer;

FIGS. 4F to 4H show the correlation between high or low serum levels of 14-3-β protein and the number of lymph nodes metastasis (FIG. 4F), the size of tumor (FIG. 4G), 14-3-3β protein level in tumor tissue (FIG. 4H) respectively before surgery;

FIGS. 5A to 5C show the correlation between high or low 14-3-3β protein values and overall survival, recurrence-free survival, and before or after the surgery in gastric cancer patients;

FIGS. 5D to 5E show the correlation between CEA values and overall survival, recurrence-free survival;

FIGS. 6A to 6E show the correlation between overexpression of 14-3-3β protein and tumor invasion, migration and growth in cells.

### DETAILED DESCRIPTION OF THE INVENTION

### Definition

The term “cancer marker”, as used herein, refers to a particular protein associated with cancer cells whose levels increased gradually during cancer progression that are released into blood from cancer cells or other cells after the induction of cancer.

The term “prognosis”, as used herein, refers to prediction of the likely progress and outcome of an illness, which includes defined outcomes (such as recovery, some symptoms, characteristics, duration, recurrence, complications, deaths, and survival rates).

The term “gastric cancer”, as used herein, refers to abnormal proliferation of mucosal cells occurring in the stomach, such as adenocarcinoma, the most common type of malignant gastric tumor.

The term “early gastric cancer”, as used herein, refers to the type of adenocarcinoma confined to the mucosa or submucosa.

The term “advanced gastric cancer”, as used herein, refers to the type of adenocarcinoma that shows invasion beyond to the submucosa or muscular layer, penetration over the gastric muscular layer (basement muscular layer), or even invasion of the adjacent lymph nodes.

The term “tumor infiltration”, as used herein, refers to the abnormal distribution of malignant tumor cells around the tissue gaps either in quantity or in quality, such as tumor cell adhesion, enzyme degradation, migration, proliferation in stroma.

The term “tumor distal metastasis”, as used herein, also called as “malignant metastasis”, refers to the migration process of tumor cells from the original site to other organs for new tumors formation through the bloodstream, or the lymph system.

The term “vascular invasion”, as used herein, refers to the condition that tumor cells and red blood cells are found in the space covered by vascular smooth muscle at the same time.

The term “organ invasion”, as used herein, refers to the condition that tumor cells invade at least one of the adjucent organs, such as duodenum, esophagus, liver, mesocolon or diaphragm.

The term “surgery”, as used herein, refers to the resection of gastric tumor with the basic goal of no remaining cancer cells. The resection scope is determined according to the location, size, and morphology of tumor.

The term “chemotherapy”, as used herein, refers to the treatment of cancer with chemical compounds to inhibit cancer growth.

The term “radiotherapy”, as used herein, refers to the treatment of cancer with ionizing radiation to kill fast growing cancer cells.

The term “staging”, as used herein, refers to the classifying process of gastric cancer for 5 stages such as stage 0 (carcinoma in situ), stage I (divided into stage IA and stage IB), stage II, stage III (divided into stage IIIA and stage IIIB) and stage V according to the tumor size, the presence of regional lymph node metastasis and distal metastasis

The term “carcinoembryonic antigen (CEA)”, as used herein, refers to the embryonic antigen found in fetal development, which is an acid glycoprotein molecule containing 40-60% sugar, with a normal range <5.0 ng/ml. Elevated CEA levels are found in a variety of cancers such as colonic, rectal, breast, gastric, pancreatic, and lung. It is used clinically to diagnose, identify, treat, or recurrence of cancers.

The term “purified”, as used herein, refers to the polypeptides or proteins that are isolated from natural source. Preferably, purified polypeptides or proteins are composed of at least 95% pure products in comparison to the natural source. Most preferably, purified polypeptides or proteins are composed of homogeneous composition. Usually 2-D gel electrophoresis separation is carried out based on the molecular weight and isoelectric point of the protein to single spots. A person skilled in the art would have known conventionally 2-D gel electrophoresis.

The term “normal sample” or “normal tissue”, as used herein, refers to normal, disease-free cell, tissue, blood or serum. The levels of 14-3-3β protein from patients are compared with that of normal sample to determine whether a patient has an excess amount. Normal sample can be obtained from the adjacent normal tissue of tumor. Preferably the level of 14-3-3β protein from normal samples is in correspondence to the patient samples, which should be obtained under the same experimental condition. Normal sample can be obtained from the same tissue or different type of tissue. Normal sample may choose from individuals with matched characteristics, such as age, sex and race. The normal levels of 14-3-3β protein in the present invention were determined, which were provided as an average range, a mean value and a standard deviation, or similar ways of expression.

The term “tissue sample” or “biological sample” or “cell or tissue from patient”, as used herein, are all similar cells collected from the tissues of patient. The sources of tissue samples can be fresh, frozen and/or stored organs or tissue samples; blood or any composition of blood, body fluids such as ascites or tissue fluids.

### Example 1

### Changes of Protein Expression in Gastric Cancer Cell Line SC-M1 after 5-FU (5-Fluorouracil) Treatment

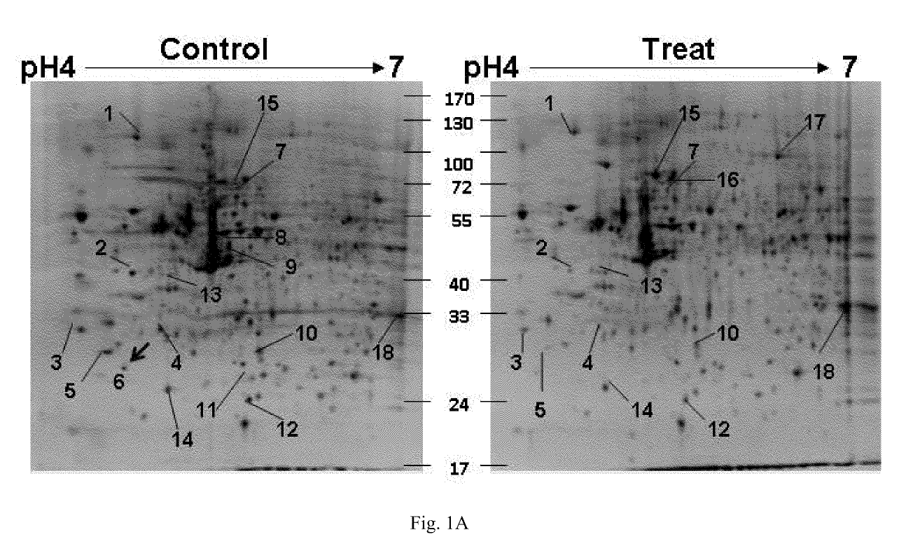

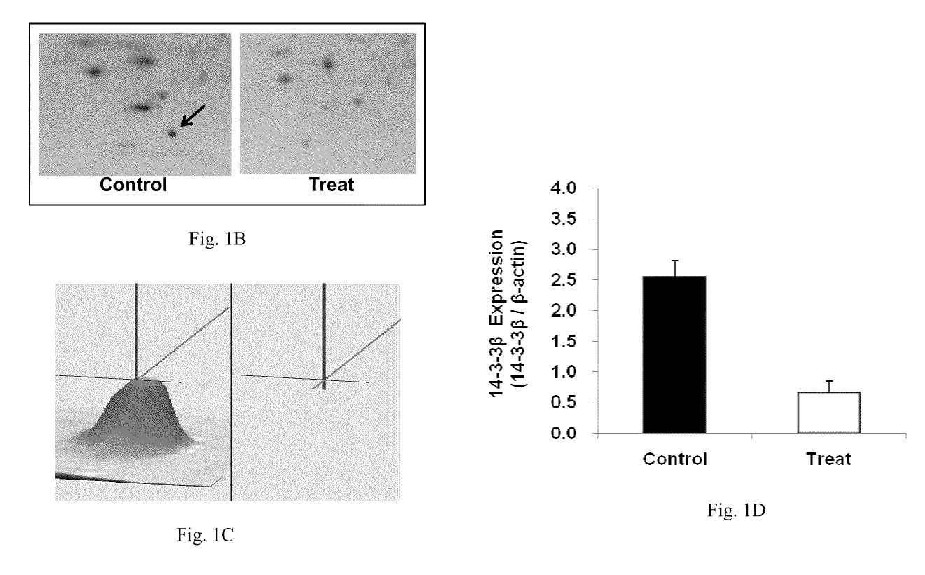

Changes of expression level of 14-3-3β protein in SC-M1 cell line were determined after 5-FU (5-fluorouracil) treatment for 48 hours using proteomics and Western blot in the present invention (FIGS. 1A-1D).

### Cell Culture

SC-M1 cells in the present invention were cultured in RPMI-1640 medium supplemented with 10% fetal bovine serum (purchased from Invitrogen Carlsbad, Calif., U.S.A.) and 100 U/ml penicillin, 100 μg/ml streptomycin and 2.5 μg/ml amphothericin (purchased from Hyclone, Logan, Utah, U.S.A) at 37° C. with 5% CO₂ and subcultured every 2-3 days.

### 2D-Polyacrylamide Gel Electrophoresis and Image Analysis

2D-gel electrophoresis was performed on 5-FU treated SC-M1 cells (the experimental group) and untreated SC-M1 cells (the control group) to compare the protein expression levels after treatment of 5-FU. 65 mM of dithioerythritol (DTE) and 0.5% (v/v) IPG buffer were added into samples of 500 μg from both groups for 1 h at room temperature and centrifuges for 30 min at 14000 g. Proteins were precipitated in IEF procedure, then using a 18 cm gel strip (pH 4-7) with IPGphor (purchased from Amersham Pharmacia Biotech) at 20° C., 8000 V for a total of 91.2 KVhr. For the second-dimension separation, the strip was separated in a 12.5% SDS-polyacrylamide gel. Protein spots in gel slabs were detected and analyzed with ImageMaster software version 6.0 (purchased from Amersham Pharmacia Biotech). The intensity of each protein spot was normalized with the total amount in the gel and expressed in relative volume (% V).

### Hydrolysis on Gel Electrophoresis and NMR Analysis

The proteins were excised from the gel and analyzed via NMR spectroscopy. Protein identification was confirmed with the MASCOT database. The Mascot Score S given as −10*Log(P) would exceed the threshold for the confirmed protein (P<0.05).

The Alamar Blue assay is used to determine cell growth and survival after SC-M1 cells were treat with 5-FU. The survival rate was significantly lowered after the addition of 5-FU. There were 18 protein spots showing significant difference in 2-D gel from Mascot searching after 5-FU treatment (FIG. 1A, left panel: control group, right panel: the experimental group). The functions of each corresponding protein spot were listed in Table 1.

TABLE 1

| | | | | | | | | | |
|---|---|---|---|---|---|---|---|---|---|
| | | | | Mol. | | | | | |
| Protein | | | | Wt. | Isoelectric | Matched | Range | | Fold |
| spot | No. | Name | Score | (Da) | point(PI) | search | (%) | Function | (T/C) |

Negative control

| | | | | | | | | | |
|---|---|---|---|---|---|---|---|---|---|
| 1 | P14625 | Endoplasmin | 229 | 92411 | 4.76 | 5 | 9 | Anti- | 0.997 |
| | | precursor | | | | | | apoptosis | |
| 2 | P08865 | 40S ribosomal | 252 | 32702 | 4.79 | 4 | 16 | Cell | 0.262 |
| | | protein SA | | | | | | adhesion | |
| 3 | Q07021 | complement1, | 155 | 31343 | 4.74 | 2 | 12 | Immune | 0.613 |
| | | subunit Q | | | | | | response | |
| | | binding | | | | | | | |
| | | protein Mit | | | | | | | |
| 4 | P08758 | Calcium-dependent | 203 | 35783 | 4.94 | 4 | 14 | Anti- | 0.211 |
| | | phospholipids | | | | | | apoptosis, | |
| | | binding | | | | | | signal | |
| | | protein | | | | | | transduction | |
| | | Annexin A5 | | | | | | | |
| 5 | P62258 | 14-3-3 epsilon | 106 | 29155 | 4.63 | 3 | 11 | Cellular | 0.044 |
| | | protein | | | | | | signal | |
| | | | | | | | | transduction | |
| 6 | P31946 | 14-3-3 beta/ | 63 | 27934 | 4.76 | 1 | 5 | Ras signal | N/A |
| | | alpha protein | | | | | | transduction | |
| 7 | P61978 | hetero nuclear | 61 | 50944 | 5.39 | 1 | 2 | mRNA | 0.543 |
| | | ribonucleoprotein | | | | | | processing, | |
| | | K | | | | | | signal | |
| | | | | | | | | transduction | |
| 8 | P05783 | Keratin I, | 56 | 47897 | 5.34 | 2 | 5 | Apoptosis | N/A |
| | | actin filament | | | | | | negative | |
| | | 18 | | | | | | control | |
| 9 | P05783 | Keratin I, | 53 | 47897 | 5.34 | 2 | 5 | Apoptosis, | N/A |
| | | actin filament | | | | | | negative | |
| | | 18 | | | | | | control | |
| 10 | P47756 | F-actin | 109 | 31200 | 5.36 | 2 | 5 | Cell | 0.340 |
| | | capping | | | | | | migration | |
| | | protein beta | | | | | | | |
| | | subunit | | | | | | | |
| 11 | P04792 | Heat shock | 151 | 22768 | 5.98 | 2 | 13 | Cell | N/A |
| | | protein | | | | | | migration | |
| | | beta-1(HSP 27) | | | | | | | |
| 12 | P09211 | Glutathione | 290 | 23210 | 5.44 | 4 | 31 | Anti- | 0.543 |
| | | S-transferase | | | | | | apoptosis | |
| | | P | | | | | | | |

Positive control

| | | | | | | | | | |
|---|---|---|---|---|---|---|---|---|---|
| 13 | P35908 | Keratin II, | 69 | 65825 | 8.07 | 1 | 2 | Keratinocytes | 1.002 |
| | | actin- | | | | | | activation, | |
| | | filament-2 | | | | | | migration, | |
| | | | | | | | | proliferation | |
| 14 | P52565 | Rho GDP | 167 | 23193 | 5.02 | 4 | 27 | Cell | 15 |
| | | dissociation | | | | | | migration, | |
| | | inhibitor | | | | | | cell adhesion | |
| | | (GDI)-1 | | | | | | negative | |
| | | | | | | | | control | |
| 15 | P11142 | Homologous | 272 | 70854 | 5.37 | 4 | 10 | Protein binding | 1.167 |
| | | heat shock | | | | | | | |
| | | protein 71 | | | | | | | |
| 16 | P08107 | Heat shock | 254 | 70009 | 5.48 | 5 | 9 | Anti- | N/A |
| | | protein 70-1 | | | | | | apoptosis | |
| 17 | P15311 | Ezrin | 174 | 69239 | 5.95 | 5 | 7 | Actin bundle | N/A |
| | | | | | | | | formation | |
| 18 | P04406 | glyceraldehyde- | 216 | 35899 | 8.58 | 4 | 16 | Glycolysis | 1.665 |
| | | 3-phosphate | | | | | | | |
| | | dehydrogenase | | | | | | | |
| | | (GAPDH) | | | | | | | |

The main function for these proteins involves anti-apoptosis, cell migration, signal transduction, cell skeleton, cell adhesion and Ras signal transduction. Those are related to tumor formation. Among these proteins, 14-3-3β protein showed a significant inhibition by 5-FU treatment in the experimental group (refers to FIGS. 1B, 1C, 14-3-3β protein was indicated by an arrow; left panel: control group, right panel: the experimental group).

### Protein Extraction

The protein samples in the experimental group were extracted for Western blot analysis to confirm the significant inhibition of 5-FU treatment to 14-3-3β protein in gastric cancer cells SC-M1.

The cell samples were resuspended in 7M of urea, 4 M of CHAPS surfactants and 2 M of thio-urea, followed by sonication. The protein concentrations were determined with Bio-Rad kit (Hercules, Calif.) after centrifugation at 13200 g for 30 min.

### Western Blot Analysis

Protein samples were separated with sodium dodecyl sulfate polyacrylamide (SDS) gel electrophoresis and transferred into a PVDF membrane (polyvinylidene difluoride membrane, purchased from Millipore Corp, Bedford, Mass.). The membrane was blocked in a blocking solution (5% nonfat dried milk in PBS) at room temperature, probed with mice anti-14-3-3 primary monoclonal antibody (Abcam, Cambridge, U.K) in a 1:1000 ratio overnight at 4 C, and then incubated with goat-anti-mice horseradish peroxidase (HRP)-conjugated anti-immunoglobulin (IgG) secondary antibody (Sigma) in a 1:8000 ratio. The signals were detected enhanced chemical luminescence (ECL) detection kit (Pierce, Boston Technology, Woburn, Mass.) and exposure to X-ray film.

Expression level with difference in folds was shown in FIG. 1D after the Western blot analysis. Expression level of 14-3-3β protein in SC-M1 cells showed a 2.55 fold difference after the treatment with 5-FU (2.55±0.27, p=0.05). Therefore 14-3-3β protein was chosen to be the detection target after the significantly inhibited expression level of 14-3-3β protein was confirmed.

### Example 2

### Difference of 14-3-3β Protein Expression in a Gastric Cancer and a Normal Tissue

The 40 stomach tissue pairs used in the example were compared from cancer with normal tissue.

### Tissue and Blood Samples

All the human samples of tissue and blood were approved by (Institution Review Board, IRB). The 40 stomach tissue pairs used in the embodiment were obtained 30 min after surgery followed by storage in liquid nitrogen. Mucosal samples were obtained 3 cm away from the tumor edge.

### Protein Extraction and Western Blot Analysis

All the tissue samples were stored in liquid nitrogen after removal. The samples were dried at −50° C. and ground in liquid nitrogen for homogeneity. The proteins were extracted, ultra sonicated, centrifuged and concentration determined All the proteins from the 40 pair samples were analyzed with Western blot.

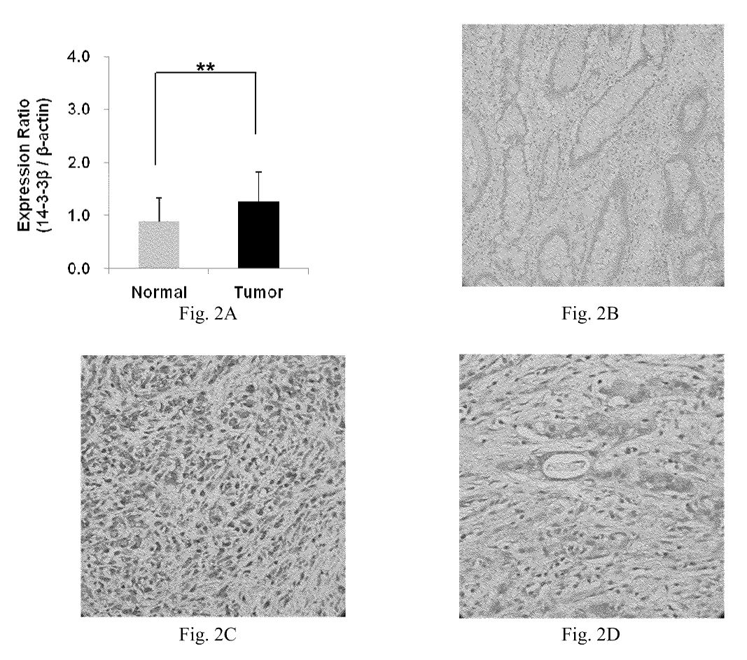

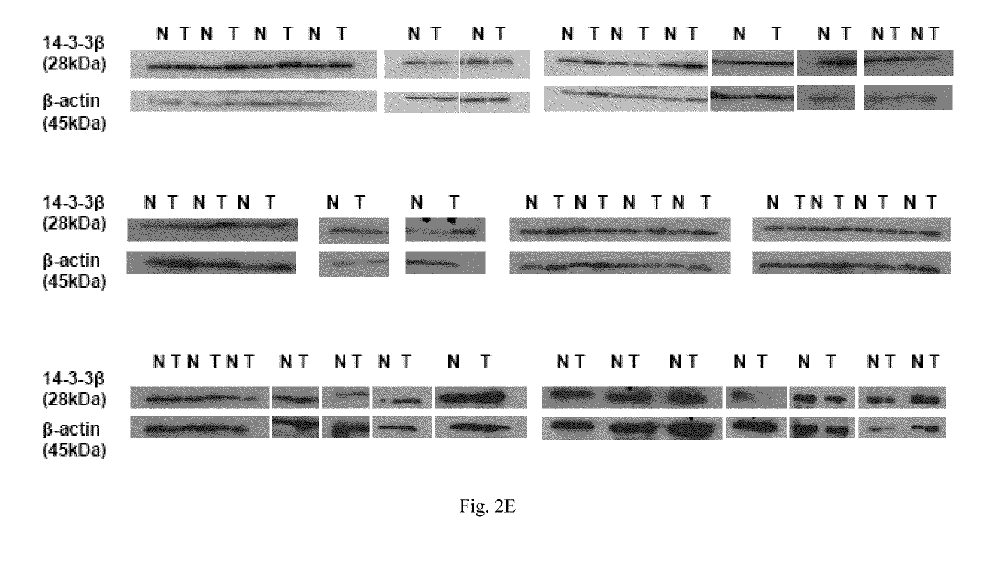

Results were shown in FIG. 2A and FIG. 2E. The Western blot analysis on 14-3-3β protein from tumor tissue (T) and normal tissue (N) was shown in FIG. 2E. The results were quantified, normalized with β-actin and showed in FIG. 2A. The 14-3-3β proteins showed a significantly higher level in cancer tissue than in normal tissue (p=0.03).

### Immunostaining of Tissue

Paraffin-embedded tissue sections in slides were rehydrated and the endogenous peroxidase activity was blocked with 3% hydrogen peroxide. The sample was blocked with horse serum in a ratio of 1:20 for 20 min at room temperature. The slide was probed with mice-anti-human 14-3-3β monoclonal antibody (Santa Cruz Biotechnology, Santa Cruz, Calif.) in a 1:100 ratio overnight at 4° C., and then incubated with donkey-anti-mice horseradish peroxidase (HRP)-conjugated anti-immuno-globulin (IgG) secondary antibody (Santa Cruz Biotechnology) at room temperature for 30 min after three time washes of PBS. The slide was stained with diaminobenzidene (DAB) followed by counter-staining of Hematoxylin, rinsed and dehydrated, then fixed with xylene.

FIGS. 2B-2D showed the immunostaining results at 200-fold magnification of normal tissue (FIG. 2B), intestinal type tumor tissue (FIG. 2C) and mixed type tumor tissue (FIG. 2D). 14-3-3β protein showed positive stain in nucleus and cytoplasm of cancer cells. Little 14-3-3β protein was seen in normal stomach tissue (FIG. 2B). Expression levels of 14-3-3β protein were significantly higher in intestinal type tumor tissue (FIG. 2C) and mixed type tumor tissue (FIG. 2D). The increased expression levels of 14-3-3β protein in cancer tissues were confirmed again in the immunostaining results.

### Example 3

### Expression Levels of 14-3-3β Protein in Gastric Cancer Patients and Normal Individuals

### Blood Sample

The blood samples in the present invention were obtained after the agreement of patients. There were 145 gastric cancer patients and 63 normal control individuals (38 males and 25 females, with an average age of 46.2 years), including 19 patients with gastritis diagnonized by endoscopy, 19 non-gastritis dyspepsia patients, and 25 volunteers without any stomach syndromes. The samples were collected and stored at −20° C. in the embodiment. All the 145 patients in the study underwent surgery to remove their stomachs during July 2001 to March 2006. The distribution of the subjects studied was shown in Table 2.

TABLE 2
Statistic summary of the gastric cancer subjects

| | Number of Patients | % |
|---|---|---|
| age | | |

| | |
|---|---|
| median | 67 |
| range | 29-89 |

| | | |
|---|---|---|
| Gender | | |
| Male | 89 | 61 |
| female | 56 | 39 |
| Staging | | |
| I&II | 60 | 41 |
| III&IV | 85 | 59 |
| Depth of tumor invasion | | |
| T1 | 17 | 12 |
| T2 | 42 | 29 |
| T3 | 80 | 55 |
| T4 | 6 | 4 |
| Metastasis of lymph nodes | | |
| N0 | 42 | 29 |
| N1 | 63 | 43 |
| N2 | 30 | 21 |
| N3 | 10 | 7 |
| Distal metastasis | | |
| M0 | 120 | 83 |
| M1 | 25 | 17 |
| Lauren classification | | |
| diffuse type | 75 | 52 |
| intestinal type | 68 | 48 |
| Organ invasion | | |
| Negative | 106 | 73 |
| Positive | 39 | 27 |
| Blood invasion | | |
| Negative | 30 | 23 |
| Positive | 100 | 77 |
| Peritoneal invasion | | |
| Negative | 124 | 86 |
| Positive | 21 | 14 |

The classification used here is the TNM staging system. The standards in the surgery of gastric cancer include complete removal of the primary tumor by surgery, partial lymph node dissection (D2), and no existence of obvious tumor. In addition, these patients showed no distal metastasis in organs such as liver, lung, distal organs and so on; no other or combined tumors, and not receiving any chemotherapy or radio therapy. The clinical pathological factors include age, gender, tumor type (Bormann classification), tumor tissue classification (Lauren classification), tumor invasion depth, lymph node metastasis, blood invasion and tumor size (length and width). All these factors and notes were recorded in the patient database. Follow-up status of the patients was determined from 3 to 46 months after surgery. Survival rates were determined in the follow-up periods. The levels of 14-3-3β protein were evaluated with the abovementioned clinical results.

### Enzyme-Linked Immunosorbent Assay (ELISA)

The amount of 14-3-3β protein in serum samples was determined with ELISA. Monoclonal antibodies against 14-3-3β protein (4 μg/ml, Abcam, Cambridge, U.K.) were put into a 96-well plate coated with streptavidin and incubated overnight at 4° C. The plate was blocked with 5% skim milk for 2 h in PBS after wash. Standard 14-3-3β protein antigen and all serum samples (diluted in a ratio of 1:400) were added into the plate for 3 h incubation. Polyclonal antibodies against 14-3-3β protein (0.08 μg/ml, Upstate Biotechnology, Inc., Lake Placid, N.Y.) were added and treated for 2 h. HRP-conjugated goat anti-rabbit IgG polyclonal antibodies (0.04 μg/ml) were added to detect antigen-antibody complexes. HRP substrate, TMB (Bionova Biotechnology, Dartmouth, NS, Canada), was added for 30 min followed by addition of 2M sulfuric acid to stop the color development. The plate was read at 450 nm with reference at 570 nm in an ELISA reader to determine the concentrations of 14-3-3β protein and unknown proteins.

### Serum Level of Carcinoembryonic Antigen (CEA)

The serum levels of CEA were measured using commercial ELSA 2-CEA kits (CIS bio international, France). ELSA2-CEA is a solid phase two-site immunoradiometric assay. Monoclonal antibodies were prepared against sterically remote antigenic sites on the CEA. The first clone binds the ELSA solid phase, and the second one, radiolabeled with 1-125, was used as a tracer. CEA ranged from 0.4 ng/ml to 0.64 ng/ml among the examined subjects in the study (control group).

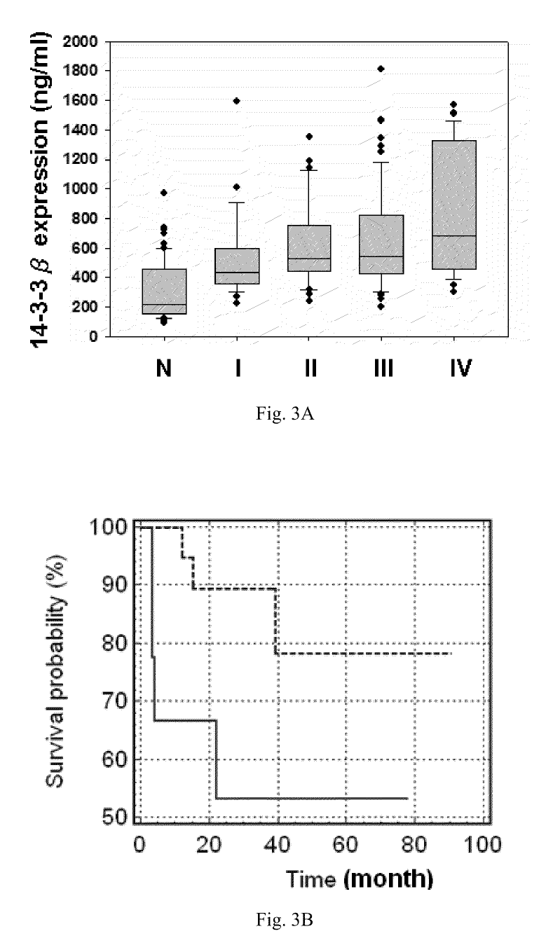

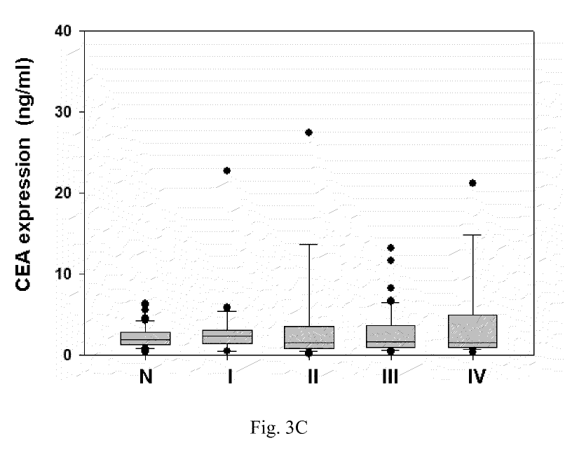

Referring to FIG. 3A and FIG. 3C, blood levels of 14-3-3β protein (FIG. 3A) and CEA (FIG. 3C) in 145 patients (stage Ito IV) and 63 normal individuals were analyzed with ELISA. The mean of plasma 14-3-3β protein value was 506 ng/ml, with a median of 424 ng/ml (n=23) in stage I patients. The plasma 14-3-3β protein levels were significantly higher in stage I patients than in normal individuals (p=0.05), while no significant difference in CEA levels (p=0.297).

Referring to FIG. 3B, the correlation between 14-3-3β protein values and survival rates in gastric cancer patients was studied. The patients were divided into 2 groups (high-14-3-3β protein group and low 14-3-3β protein group) when the mean 14-3-β protein value (521 ng/ml) was used as the cutoff value (n=28). As shown in the figure, patients with high 14-3-3β protein levels (solid line) showed lower survival rates (p=0.039) than patients with low 14-3-3β protein levels (dashed line).

### Example 4

### The Correlation Between 14-3-3β Protein or CEA and Patients of Each Stages and Normal Individuals

The patients were divided into different groups according to clinical and pathological parameters to analyze the correlation of 14-3-3β protein and clinical outcome. The difference between two independent groups was compared with Student's t test. There is statistic significance if P<0.05. One-way ANOVA (SAS 9.1 software) was applied to compare the difference among multiple groups. The correlation of variables was analyzed using MedCalc software version 9.0, and the correlation coefficient was represented by P-value.

Receiver operating characteristic curve (ROC) analysis has been extensively used in the evaluation of diagnostic test as a description of diagnostic accuracy. Global measure of the accuracy of a diagnostic tool is the area under the curve (AUC). AUC was used to statistically compare the area under two ROC curves. The AUC value (larger than 0 and smaller than 1) of a diagnostic test denotes whether or not a patient has a disease under the two alternative-forced choice condition. The levels of 14-3-3β protein and CEA in normal individuals or patients in different stages were plotted with sensitivity versus specificity to determine the area under ROC curve in the embodiment of the present invention.

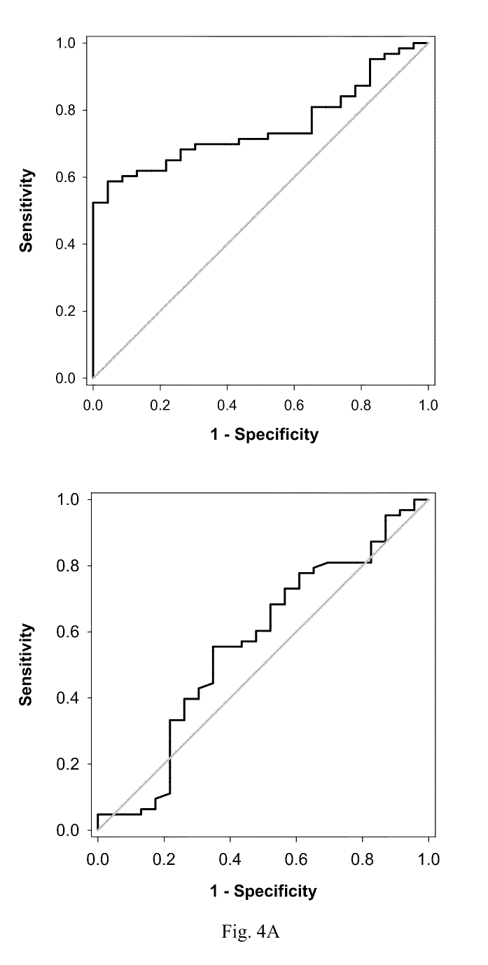

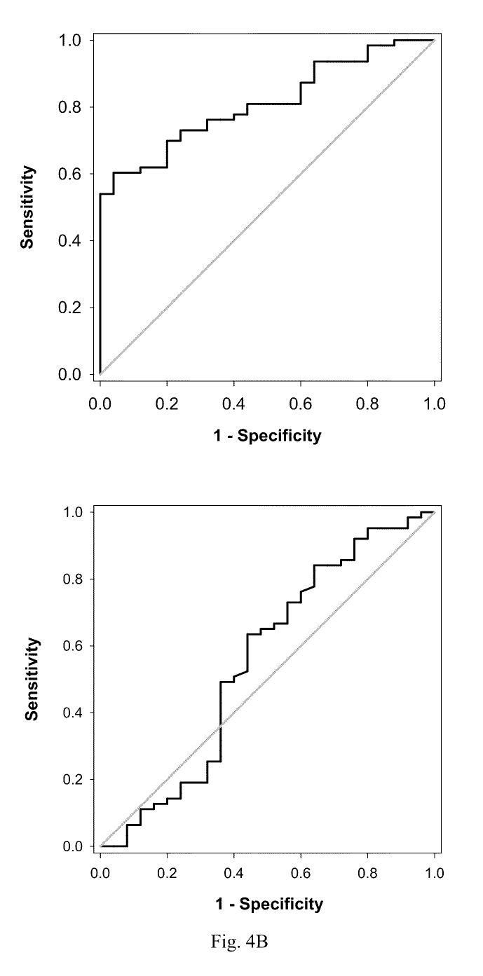

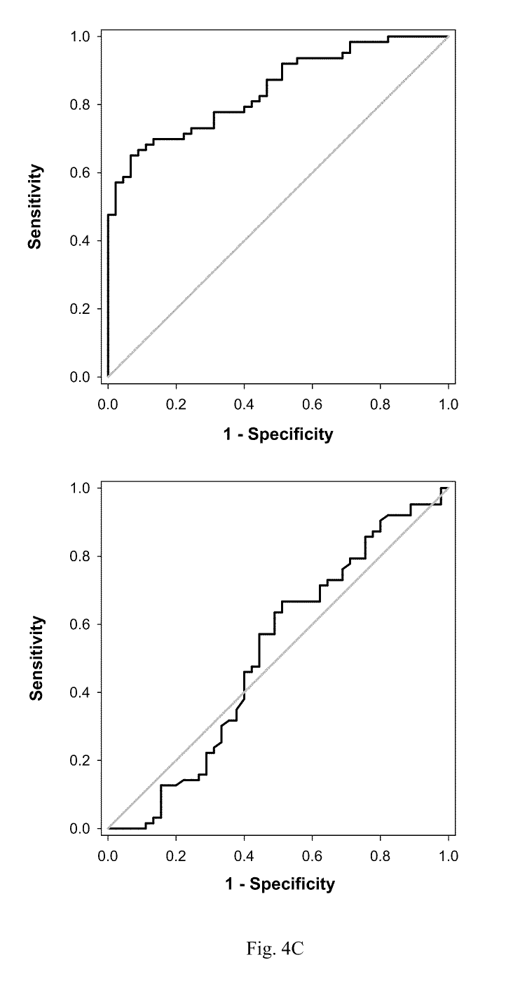

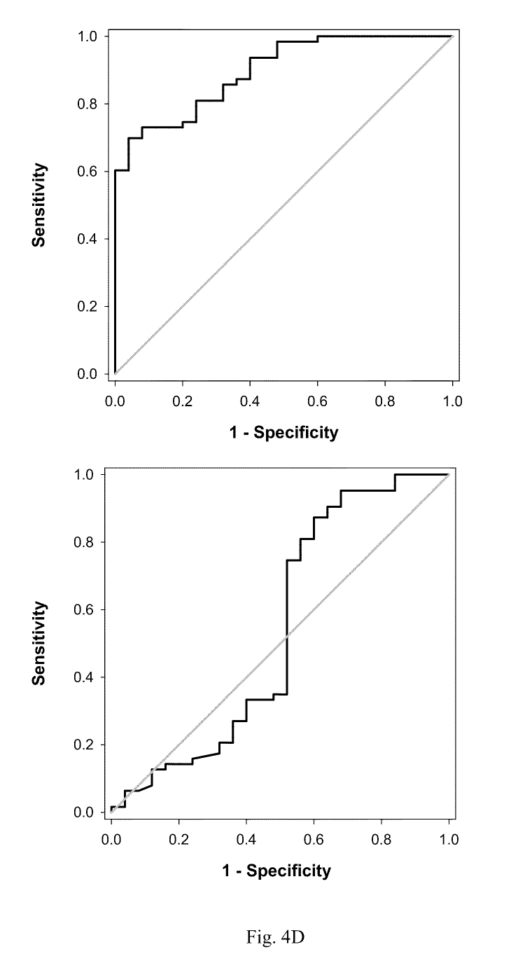

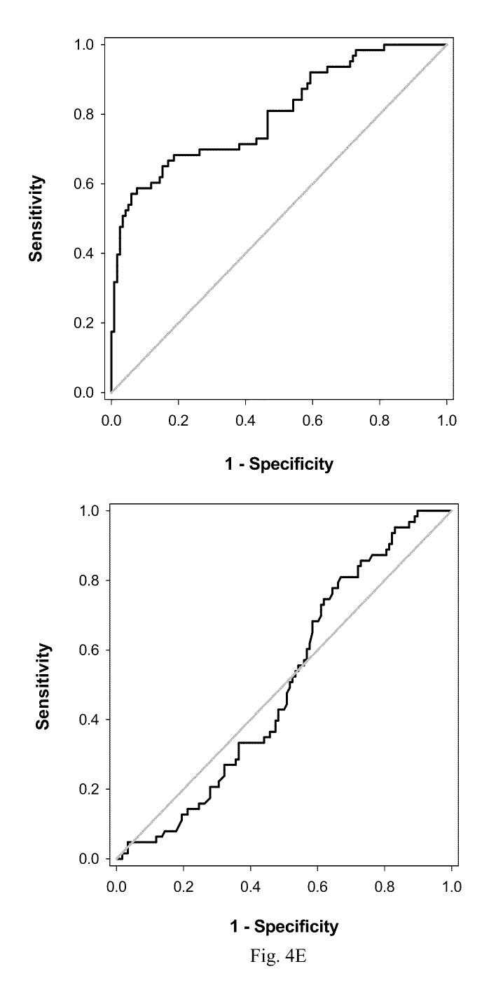

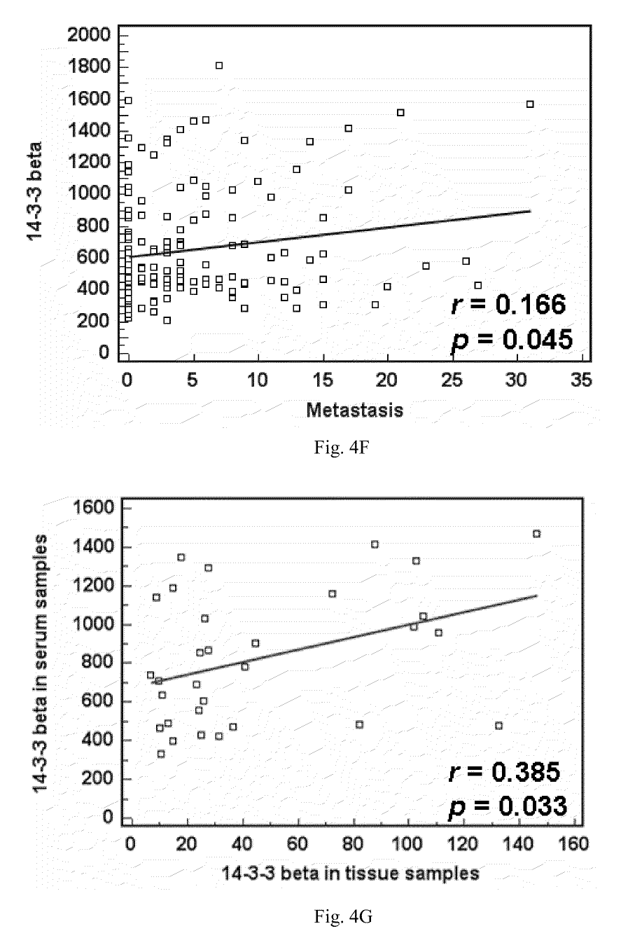

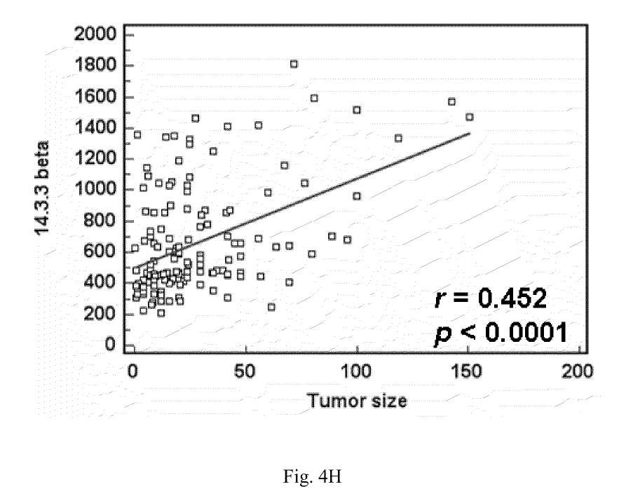

Referring to FIGS. 4A to 4E, FIG. 4A shows the ROC curves of 14-3-3β protein and CEA in patients with stage I gastric cancer. The AUC value of levels of 14-3-3β protein in the stage I cancer patient was 0.75. The sensitivity was 82% and specificity was 65% with the optimal cut-off point 329 ng/ml for early detection of gastric cancer. This result indicates that 14-3-3β protein is an ideal early gastric cancer marker. In addition, AUC values of 14-3-3β protein were higher than CEA in all patients with various stage of gastric cancer, which showed that the detection of 14-3-3β protein is more accurate than CEA and therefore is a better diagnostic marker.

Referring to FIGS. 4F to 4H, the correlation of serum levels of 14-3-3β protein and the number of lymph node metastasis (FIG. 4F), the size of tumor (FIG. 4G), 14-3-3β protein level in tumor tissue (FIG. 4H) respectively before surgery were shown. The level of 14-3-3β protein and the number of lymph node metastasis (r=0.166, p=0.045) and the size of tumor (r=0.452, p<0.001) showed a high correlation. In addition, the serum level of 14-3-3β protein and the 14-3-3β protein levels in tumor tissue were closely related before surgery (r=0.385, p=0.033). On the other hand, as shown in Table 3, the serum level of 14-3-3β protein had a higher correlation to late stage of gastric cancer (p=0.002), tumor invasion depth (p=0.008), and peritoneal invasion (p=0.045) respectively but lower correlation to Lauren classification (p=0.831), organ invasion (p=0.157) and blood invasion (p=0.137). These results indicated that 14-3-3β protein may play an important role in invasion mechanism of the gastric cancer.

TABLE 3
The correlation between 14-3-3β, CEA and clinical factors

| | 14-3-3β | | | CEA | | |
|---|---|---|---|---|---|---|
| | Mean | Median | p | Mean | Median | p |
| Staging | | | 0.002* | | | 0.206 |
| I/II (N = 48) | 545.95 | 460.22 | | 4.95 | 2.02 | |
| III/IV (N = 70) | 736.09 | 628.00 | | 27.14 | 1.56 | |
| Depth of tumor invasion | | | 0.008* | | | 0.767 |
| T1 (N = 14) | 468.47 | 434.39 | | 4.41 | 2.88 | |
| T2 (N = 35) | 607.20 | 460.50 | | 4.73 | 1.56 | |
| T3 (N = 66) | 736.56 | 651.33 | | 28.54 | 1.53 | |
| T4 (N = 3) | 453.65 | 447.17 | | 8.95 | 4.25 | |
| Metastasis of lymph nodes | | | 0.406 | | | 0.744 |
| N0 (N = 33) | 591.53 | 474.39 | | 3.45 | 2.14 | |
| N1 (N = 51) | 650.25 | 532.17 | | 38.25 | 1.95 | |
| N2 (N = 25) | 678.08 | 588.28 | | 2.15 | 1.40 | |
| N3 (N = 9) | 899.61 | 853.28 | | 2.19 | 1.06 | |
| Lauren classification | | | 0.831 | | | 0.129 |
| Diffuse type (N = 60) | 655.80 | 532.72 | | 2.33 | 1.34 | |
| Intestinal type (N = 57) | 669.27 | 554.39 | | 35.04 | 2.53 | |
| Organ invasion | | | 0.157 | | | 0.429 |
| Negative (N = 87) | 629.19 | 517.17 | | 10.74 | 1.73 | |
| Positive (N = 31) | 741.69 | 631.61 | | 38.81 | 1.90 | |
| Blood invasion | | | 0.137 | | | 0.182 |
| Negative (N = 24) | 596.36 | 481.89 | | 4.32 | 2.80 | |
| Positive (N = 81) | 710.37 | 588.28 | | 24.55 | 1.54 | |
| Peritoneal invasion | | | 0.045* | | | 0.125 |
| Negative (N = 101) | 623.13 | 517.17 | | 20.81 | 1.90 | |
| Positive (N = 17) | 870.31 | 865.5 | | 2.11 | 1.40 | |

### Example 5

### The Correlation Between 14-3-3β Protein or CEA and Survival Rate of Gastric Cancer Patients

The Kaplan-Meier overall and recurrence-free survival curves were constructed to estimate serum level of 14-3-3β protein and survival rates. Significance was accepted for a p-value less or equal to 0.05. Paired Student's t-tests were used to compare the serum levels of 14-3-30 protein before and after surgery.

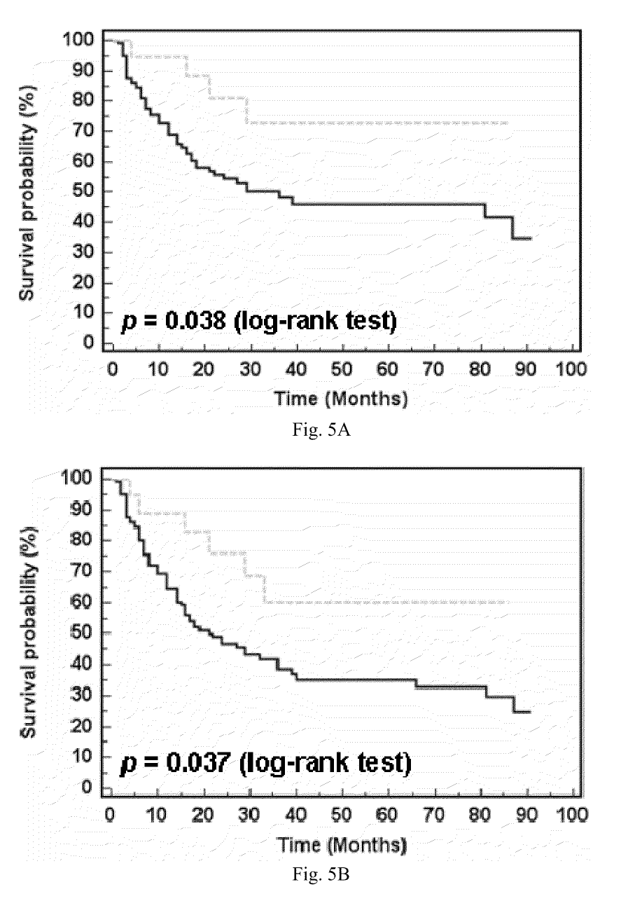

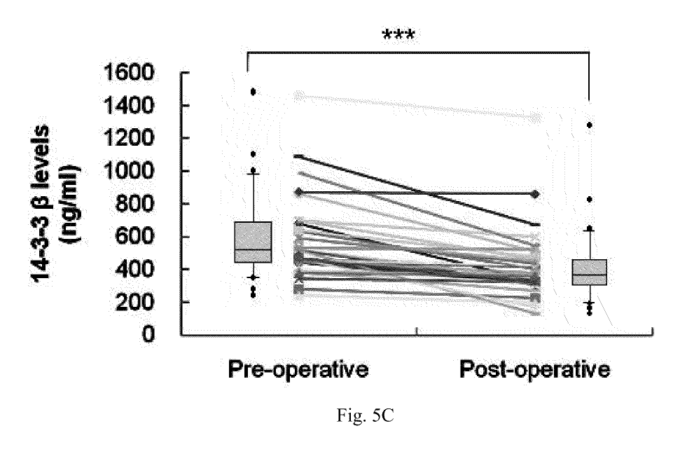

Referring to FIG. 5A to FIG. 5B, the correlation between 14-3-β protein values and survival rates in gastric cancer patients was studied. The patients were divided into 2 groups (high-14-3-3β protein group and low 14-3-3β protein group) when the 14-3-3β protein value (349 ng/ml) with high sensitivity (86%) and high specificity (67%) was set as cutoff value. The overall survival and recurrence-free survival were compared in these 2 groups. Patients in high-14-3-3β protein group were shown to have a poor survival rate either in overall survival (FIG. 5A, p=0.038) or in recurrence-free survival (FIG. 5B, p=0.037).

On the other hand, FIG. 5C showed the plasma 14-3-3β protein levels before or after the surgery in gastric cancer patients. Significantly higher levels of 14-3-3β protein were shown in patients before surgery (mean: 579 ng/ml; median: 515 ng/ml) than those after surgery (mean: 427 ng/ml; median: 378 ng/ml).

Referring to FIG. 5D to FIG. 5E, the correlation between CEA values and the overall and recurrence-free survival rates in gastric cancer patients was shown. Relatively speaking, patients with CEA values higher than 3 ng/ml showed no correlation with the overall and recurrence-free survival. This result indicated that 14-3-3β protein is a better tumor marker than CEA.

### Example 6

### Overexpression of 14-3-3β Protein and its Relation to Tumor Invasion, Migration and Growth

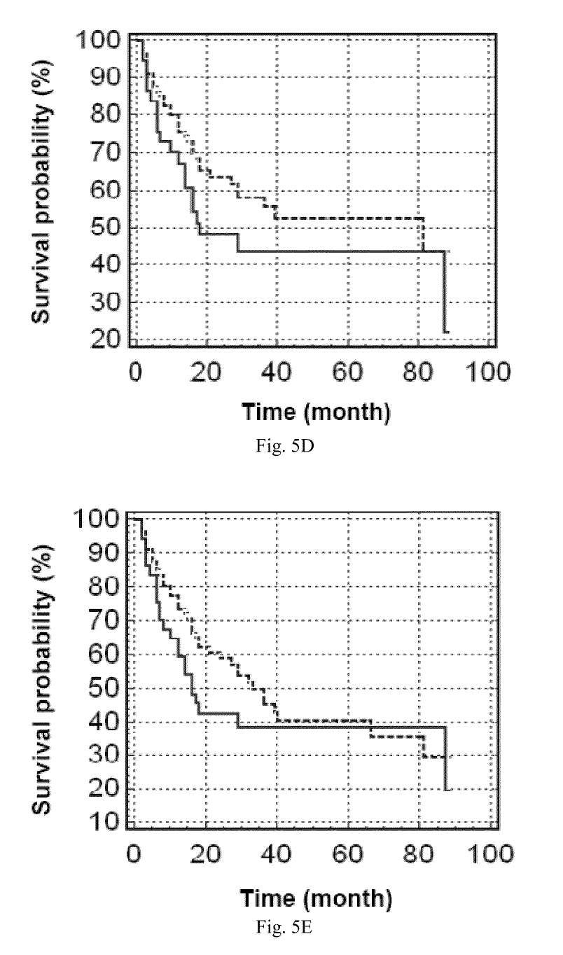

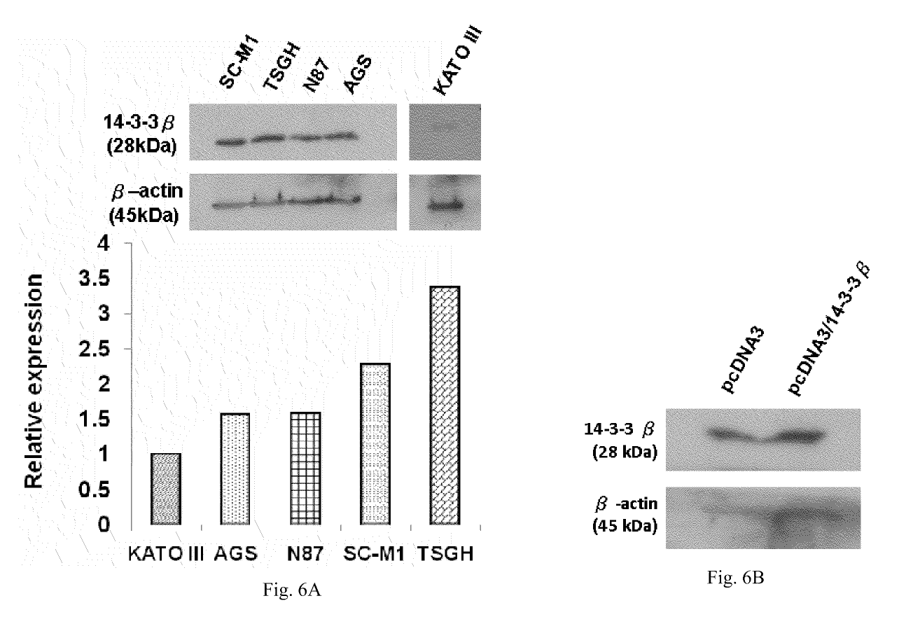

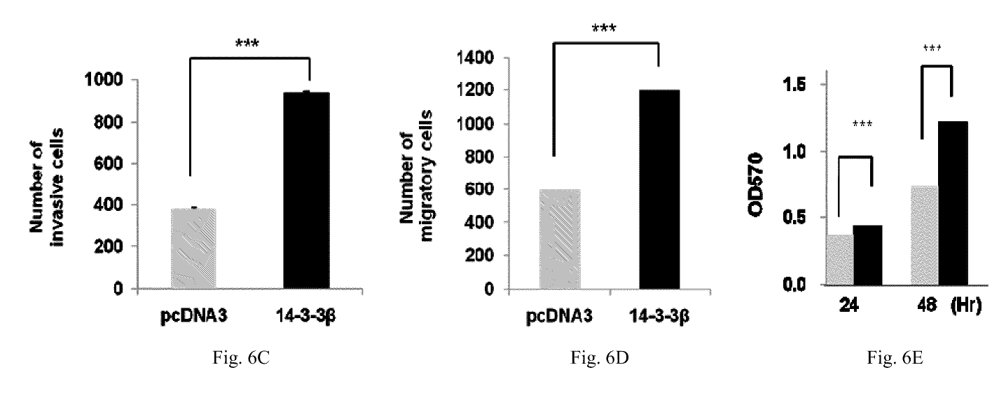

Gastric cancer cell lines TSGH, SC-M1, N87, AGS and KATO III were cultivated and analyzed through Western blotting as described. The endogenous expression of 14-3-3β protein in different gastric cancer cell lines was shown in FIG. 6A. Among them, TSGH cells showed the highest expression level of 14-3-3β protein.

### Construction of Plasmid Containing 14-3-3β

Total RNA was extracted from gastric cancer cell lines TSGH to synthesize cDNA and amplified with reverse PCR using the forward primer (5′-GGTACGTAAGCTTGCCACCATGACAATGGATAAAAGT-3′) and the reverse primer (5′-AGTCGAGAATTCTTAGTTCTCTCCCTCCCC-3′). The product was inserted into pcDNA vector (Invitrogen Inc.) to generate pcDNA3/14-3-3β plasmid for transient and permanent transfection in human gastric cancer cell lines.

### Plasmid Transfection

6×10⁵ of AGS cells were cultivated in 6-well plates at 37° C. for 24 h. Transduction of 14-3-3β containing plasmid and control plasmid were performed in AGS cells. All transfections were performed in OPTI-MEM. Plasmid DNA of pcDNA3/14-3-3β or pcDNA3 (8 μg) and 10 μl of Lipofectamine 2000 were mixed and transfected according to the manufacturer's instructions. Expression levels of 14-3-3β protein in pcDNA3/14-3-3β plasmid or pcDNA3 plasmid transfected cells were shown in FIG. 6B. The level of 14-3-3β protein was about 2-fold in pcDNA/14-3-3β plasmid transfected cells than in pcDNA3 control plasmid transfected cells.

Cell Migration and Invasion Assays with Boyden Chamber System

Expression levels of 14-3-3β protein were evaluated and studied using adapted Boyden chamber and PVPF (diameter: 8 μm) for cell migration and using BD BioCoat Matrigel (BD Biosciences) for cell invasion. 2.5×10⁴ cells in 100 μl of culture medium were seeded into the upper wells. The cells were observed under a microscope (Olympus) and counted 48 hours after adhesion and staining.

The results were summarized in FIG. 6C and FIG. 6D. The invasion and migration abilities were enhanced in the cancer cell after the plasmid containing 14-3-313 was overexpressed in transfected cells.

### Cell Proliferation Determined by MTT Assay

MTT assay is commonly used to determine cell proliferation, percent of viable cells, and cytotoxicity. 3-[4,5-dimethylthiazol-2-yl]2,5-diphenyltetrazolium bromide (MTT) is a yellow dye, which can be absorbed by the living cells and be reduced to insoluble and purplish blue formazan crystals by succinate tetrazolium reductase in mitochondria. Formazan formation can therefore be used to assess and determine the proliferation of cells.

Cells (5×10⁴) were cultivated in a 24-well plate for 24 h. The cells were transfected with plasmid containing 14-3-3β or the control plasmid for 24 and 48 h. MTT was added the amount of 100 μg/ml and incubated for 4 h at 37° C., followed by addition of DMSO (500 μl/well) to solubilize the formazan and standing for 10 min The plates were read on an ELISA reader at wavelength of 570 nm for 4 times in each experimental group.

The results were summarized in FIG. 6E. The cell growth abilities were enhanced in the cancer cell after the plasmid containing 14-3-3β was transfected. Overexpression of 14-3-3β protein could promote the gastric cancer cell growth.

According to the abovementioned illustration and examples, expression levels of 14-3-3β protein were higher in gastric cancer tissue samples than in normal tissues. Similar results were shown in the blood samples. The plasma 14-3-3β protein levels in gastric cancer patients were higher than the normal individuals, and it was increased with tumor stage in patients with gastric cancer. From the view of prognosis, the patients with higher 14-3-3β protein levels after surgery showed poor prognosis and lower survival rate. These results suggest that 14-3-3β protein is not only a highly cancer-related protein, but also a tumor marker which can be applied in early diagnosis and prognosis for gastric cancer. Therefore, a detecting kit containing valid amount of anti-14-3-3β protein antibodies base on the abovementioned method can be applied in detecting the risk for the development of tumor invasion or distal metastasis from gastric cancer

These above examples should not, however, be considered to limit the scope of the present invention, it is contemplated that modifications will readily occur to those skilled in the art, which modifications will be within the spirit of the invention and the scope of the appended claims.

What is claimed is:

1. A method for determining the prognosis of gastric cancer, comprising:
providing a biological sample;
determining an expression level of 14-3-3β protein in the biological sample; and
comparing the expression level of 14-3-3β protein in the biological sample with a reference expression level of 14-3-β protein in a normal sample;
wherein a poor prognosis is indicated if the expression level of 14-3-3β protein in the biological sample is higher than the reference expression level of 14-3-3β protein of the normal sample.

2. The method of claim 1, wherein the biological sample is blood or a tissue.

3. The method of claim 1, wherein the biological sample is serum.

4. The method of claim 1, wherein the gastric cancer is gastric adenocarcinoma.

5. The method of claim 1, wherein the expression level of 14-3-3β protein in the biological sample is determined with a specific antibody of 14-3-3β protein.

6. The method of claim 1, wherein the expression level of 14-3-3β protein in the biological sample is determined by Western blot analysis.

7. The method of claim 1, wherein the biological sample is taken from a patient after a gastric cancer therapy.

8. The method of claim 7, further comprising detecting a survival rate of the patient.

9. The method of claim 8, wherein the gastric cancer therapy is surgery.

10. The method of claim 8, wherein the gastric cancer therapy is radiotherapy.

11. The method of claim 8, wherein the gastric cancer therapy is chemotherapy.

12. A method of screening a compound for inhibiting the expression of 14-3-3β protein in a biological sample, comprising:
contacting a test compound with a biological sample expressing 14-3-3β protein;
detecting the expression level of 14-3-3β protein in the biological sample; and
selecting the test compound that inhibits the expression level of 14-3-3β protein in the biological sample as compared to the expression level of 14-3-3β protein detected in the absence of the test compound.

13. The method of claim 12, wherein the biological sample is blood or a tissue.

14. The method of claim 12, wherein the biological sample is serum.

15. The method of claim 13, wherein the tissue is a stomach tissue.

16. The method of claim 15, wherein the stomach tissue is taken from a gastric cancer subject.

17. A kit comprising an effective dose of an anti-14-3-3β protein antibody for detecting the risk for developing tumor invasion or distal metastatsis of gastric cancer.

18. The kit of claim 17, wherein the gastric cancer is early gastric cancer.

19. The kit of claim 17, wherein the gastric cancer is gastric adenocarcinoma.
